(19) United States
(12) Patent Application Publication
(10) Pub. No.: US 2010/0051037 A1
(43) Pub. Date: Mar. 4, 2010

(54) CLOTHES AND METHOD FOR PREVENTING SLEEPING RESPIRATORY OBSTRUCTION AND/OR DECUBITUS

(75) Inventors: Junghwa Hong, Anyang-si (KR); Chol Shin, Seoul (KR)

Correspondence Address: David A. Einhorn, Esq.;Baker & Hostetler LLP, 45 Rockefeller Plaza, New York, NY, 10111, US

(73) Assignee: BIO SLEEP MED Co., Ltd., Seoul (KR)

(21) Appl. No.: 12/615,568

(22) Filed: Nov. 10, 2009

(30) Foreign Application Priority Data
May 11, 2007 (KR) 10-2007-0046048

(51) Int. Cl. A61F 5/56; A61F 13/00

(52) U.S. Cl. 128/848; 128/889

(57) ABSTRACT

Clothes have a capability of preventing sleeping respiratory obstruction and/or decubitus, which is worn by a user. The clothes include a plurality of air chambers provided at the rear of the clothes, the air chambers being capable of being shrunk and expanded. An air supply unit supplies air to each of the air chambers, and a pressure detecting unit measures the pressure of each of the air chambers to produce a measured pressure as an electrical signal. A controller receives the measured pressure signal from the pressure detecting unit to supply the air to the air chambers by using the air supply unit when a snoring or a sleep apnea of the user is detected or a predetermined time elapses in the same posture in order to change a posture of the user.

### TECHNICAL FIELD

The present invention relates to clothes and method for preventing a sleeping respiratory obstruction and/or a decubitus; and more particularly, clothes having a capability of preventing a sleeping respiratory obstruction and/or a decubitus and a sleeping respiratory obstruction and/or decubitus prevention method using the same.

### BACKGROUND ART

In general, a habitual snoring, an obstructive sleep apnea, and an upper airway resistance syndrome classified as a sleep-disordered breathing are diseases in which the repetitive closure of the upper airway occurs during sleep. Such diseases hinder sound sleep by deteriorating sleep efficiency at night and especially decrease a blood oxygen saturation rate [see, Chrokroverty S. (1994) Sleep Disorder Medicine. Butterworth-Heinemann].

The sleep-disordered breathing causes a daytime drowsiness, a deteriorated power of concentration, a memory loss, a decreased learning ability, a chronic fatigue and the like. Further, the sleeping respiratory obstruction leads to accidents at construction sites and workplaces and traffic accidents caused by a drowsy driving, thereby inflicting social and economical damages.

There have recently been several reports on a close relationship between the sleep-disordered breathing and the occurrence of obesity, high blood pressure, diabetes, dementia, cardiovascular diseases, cardiac paralysis, sexual function decline, cerebrovascular diseases, paralysis and metabolic syndrome [see, Prospective study of the association between sleeping respiratory obstruction and hypertension. N Engl Med 2000; 342: 1378-1384].

An upper airway closure of a human body like this will be explained in the following with reference to the accompanying drawings.

As shown in FIG. 1, an upper airway 4 for introducing air into a bronchus and a lung (all not shown) is sufficiently secured in a normal state. However, referring to an obstructive sleep apnea state illustrated in FIG. 2, a soft tissue 6 extended from a back part of a palate 8 is pressed by a self-weight and a weight of a tongue 7, thereby closing the upper airway 4.

Sleeping in a supine position worsens the closure of the upper airway 4. Further, such closure of the upper airway 4 leads to an obstructive sleep apnea in which breathing stops or is disrupted during sleep, and snoring occurs when the upper airway 4 is partially closed during sleep.

Various methods have been attempted to treat the snoring or the sleep apnea.

In general, there have been attempts of treating the sleeping respiratory obstruction by use of a functional pillow sheet during sleep, and there have been developed pillow sheets which prevent the sleeping respiratory obstruction through a posture correction by mainly elevating a lateral position or a head position.

Recently, a memory foam pillow sheet made of polymer foam developed by NASA has been commercially sold while claming as the pillow sheet for preventing a sleeping respiratory obstruction. Although such a memory foam pillow sheet is ergonomically designed to absorb load and shock transmitted to a human body and return to an original shape after the load is released, it does not bring a significant improvement for the treatment of a snoring or a sleep apnea.

Many apparatuses and methods have been developed for solving such problems. One of the apparatuses is disclosed in a utility model No. 20-0236225 which is entitled an “APPARATUS OF DRIVING A SNORING PREVENTION PILLOW SHEET BY SENSING SNORING”, and is registered in the Korean Intellectual Property Office, as shown in FIG. 3. The utility model is commonly assigned to Applicant of this present application.

The snoring prevention apparatus includes a snoring signal sensing unit 10 which inputs snoring and various other noises and outputs a desired snoring signal; a controller 30 which analyzes the signal applied from the snoring signal sensing unit 10 and starts a rotary motor driver 40 if the applied signal is confirmed as a snoring signal; a memory which stores the signal applied from the controller 30 or outputs to the controller 30; and a rotary memory driver 40 where a rotary motor 42 is driven by the signal applied from the controller 30 to drive a posture change plate 46, thereby changing the shape of a pillow sheet. The controller 30 processes the snoring signal and the noise signal applied from the snoring signal sensing unit 10 to obtain a final data and calculate a time duration when the final data continuously has a value which is larger than a threshold value. The controller 30 calculates the number of maximum points which exist for the duration if the duration is higher when compared with a set-time. The controller 30 determines the signal as the snoring signal if the number of maximum points is higher than a set value for maximum points. Further, the controller 30 applies drive signals to the rotary motor driver 40 if the snoring signal is sensed as many times as the number of the set value to drive the rotary motor 42.

Further, a utility model No. 20-0395890, which is entitled “APPARATUS FOR REDUCING SNORING” as shown in FIGS. 4 to 6 and is registered in the Korean Intellectual Property Office. The utility model is commonly assigned to Applicant of this present application.

The snoring prevention apparatus includes an air pad 71 which detects the breath of a sleeper and is expanded by the air injected thereinto when snoring is sensed, thereby correcting the sleep posture of the sleeper; an air tube 72 which is combined with the air pad 71 to transmit the detected breathing to the next stage and to act as a passage of injecting and discharging air; a sensor for converting the breathing transmitted to the air tube 72 into an electrical signal; a preamplifier which pre-amplifies the breathing sense signal outputted from the sensor; a band pass filter unit which filters the breathing sense signal outputted from the preamplifier for each band and outputs it as a heart beat/breathing/snoring determining signal; a function input unit for inputting function and mode conversion signals; a central processing unit 73 which determines whether or not there is snoring based on the signal obtained by the preamplifier and the band pass filter unit in response to the signal inputted from the function input unit, which controls to display a breathing state, generates an air injection signal for expanding the air pad if it is determined as snoring, and generates a control signal to discharge the air injected into the air pad after the set time if no snoring is sensed after the air pad is expanded; a display which displays on a screen the breathing state, the heart beat status and the like of the sleeper in accordance with the state display control signal transmitted from the central processing unit 73; a driver for outputting a light emitting diode control signal, the air injection control signal and a discharge control signal, which are outputted from the central processing unit, as each corresponding drive signal for each of the air injection control and the discharge control signal; a light emitting diode (LED) for visually displaying whether or not power is supplied and a function operation state in accordance with a light emitting diode drive signal outputted from the driver; a motor for driving a pump which injects air to the air pad 71 in accordance with the air injection signal outputted from the driver; and a discharge solenoid which opens and closes a discharge valve in accordance with the discharge control signal outputted from the driver.

In this way, the snoring prevention apparatus of the prior art in FIG. 3 automatically drives a snoring prevention pillow sheet to incline the sleeper to one side if snoring is sensed and determined, thereby enabling the sleeper not to snore. However, the snoring prevention apparatus according to the embodiment of the prior art cannot return to the original state from a state in which the snoring prevention pillow is inclined after sensing a snoring, unless the user manually resets the apparatus. There is a problem in operating such an apparatus reliably in that it becomes hard to prevent snoring if the sleeper moves away from the pillow sheet through movement or position change during sleep. Further, the sleeper's sound sleep might be disturbed because noise is generated due to a mechanical friction between a cam shaft and a plate.

Further, the snoring prevention apparatus of the prior art in FIGS. 4 to 6 induces posture change by use of the air pad 71, but is not an active type and could awake the sleeper by disturbing the sound sleep of the sleeper because it is a type which encourages to change himself/herself his/her postures. There also lies a problem in operating such an apparatus reliably because, in case that the sleeper moves away from the expansion portion of the air pad 71 when the sleeper moves or changes his position, even such a posture change cannot be induced.

In addition, a weak patient who is confined to bed for a long time or a patient who undergoes a cerebrospinal disorder and cannot make a posture change freely is at high risk for a decubitus.

The decubitus occurs among patients who undergo heart diseases, high fever, diabetes, and cerebrospinal disorders. Particularly, the decubitus is caused by unrelieved pressure to a body prominence, such as the hip, pelvis, and heel. That is, the decubitus occurs since the blood vessel is pressed due to the unrelieved pressure, and oxygen and nutritive elements are not supplied to the tissues.

Accordingly, when a patient cannot move freely, it is necessary to cyclically change his/her posture each time a predetermined time elapses.

However, it is difficult for someone to change the posture of the patient every time. Accordingly, in the prior art, a poromeric mattress that prevents the decubitus has been developed and placed on the market. Nevertheless, it is most preferable to prevent the decubitus by changing the posture of the patient at any time, thereby facilitating the blood circulation. As a result, there is a need for a product that can make the posture change freely.

### DISCLOSURE OF INVENTION

### Technical Problem

It is, therefore, a primary object of the invention to provide clothes and method for preventing a sleeping respiratory obstruction, which is capable of unaffectedly changing a sleep posture to stop a snoring and/or a sleep apnea without awakening the sleeper and capable of making such a posture change accurately even if the sleeper is made to move unconsciously by accurately determining whether or not the sleeper snores or undergoes a sleep apnea based on an ambient noise, thereby enabling the sleeper to carry on a sound sleep and preventing a disordered breathing, such as an obstructive sleep apnea.

It is another object of the invention to provide clothes for preventing a decubitus, which is worn by a weak patient who is confined to bed for a long time or a patient who undergoes a cerebrospinal disorder and cannot make a posture change freely, thereby changing his/her lying posture each time a predetermined time elapses and preventing a decubitus.

### Technical Solution

In accordance with an aspect of the invention, there is provided clothes having a capability of preventing sleeping respiratory obstruction and/or decubitus, which is worn by a user, the clothes comprising: a plurality of air chambers provided at the rear of the clothes, the air chambers being capable of being shrunk and expanded; an air supply unit for supplying air to each of the air chambers; a pressure detecting unit for measuring the pressure of each of the air chambers to produce a measured pressure as an electrical signal; and a controller for receiving the measured pressure signal from the pressure detecting unit to supply the air to the air chambers by using the air supply unit when a snoring or a sleep apnea of the user is detected or a predetermined time elapses in the same posture in order to change a posture of the user.

In accordance with another aspect of the invention, there is provided a sleeping respiratory obstruction prevention method, in which a user wears clothes having a plurality of shrinkable and expandable air chambers, the method comprising the steps of: supplying air for pressure measurement to the air chambers; measuring the pressure of one or more the air chambers pressurized by a weight of the user; extracting a bio-signal from the measured pressure; determining whether or not a snoring or a sleep apnea is detected in the bio-signal; and supplying the air to the air chambers to expand the air chambers if the snoring or the sleep apnea is detected in the bio-signal, to thereby change a posture of the user.

In accordance with another aspect of the invention, there is provided a decubitus prevention method, in which a user wears clothes having a plurality of shrinkable and expandable air chambers, the method comprising the steps of: supplying air for pressure measurement to the air chambers; measuring the pressure of one or more the air chambers pressurized by a weight of the user; determining, based on the measured pressure, whether or not the user is in the same posture for a predetermined time; and supplying the air to the air chambers to expand the air chambers if the user is in the same posture for the predetermined time, to thereby change a posture of the user.

### BRIEF DESCRIPTION OF THE DRAWINGS

The above and other objects and features of the present invention will become apparent from the following description of embodiments, given in conjunction with the accompanying drawings, in which:

FIGS. 1 and 2 are drawings explaining a closure of an upper airway of human body;

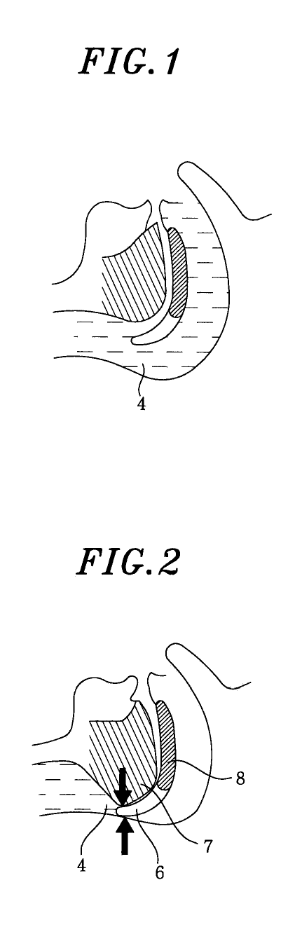

FIG. 3 is a drawing illustrating the essential parts of a prior art snoring prevention apparatus;

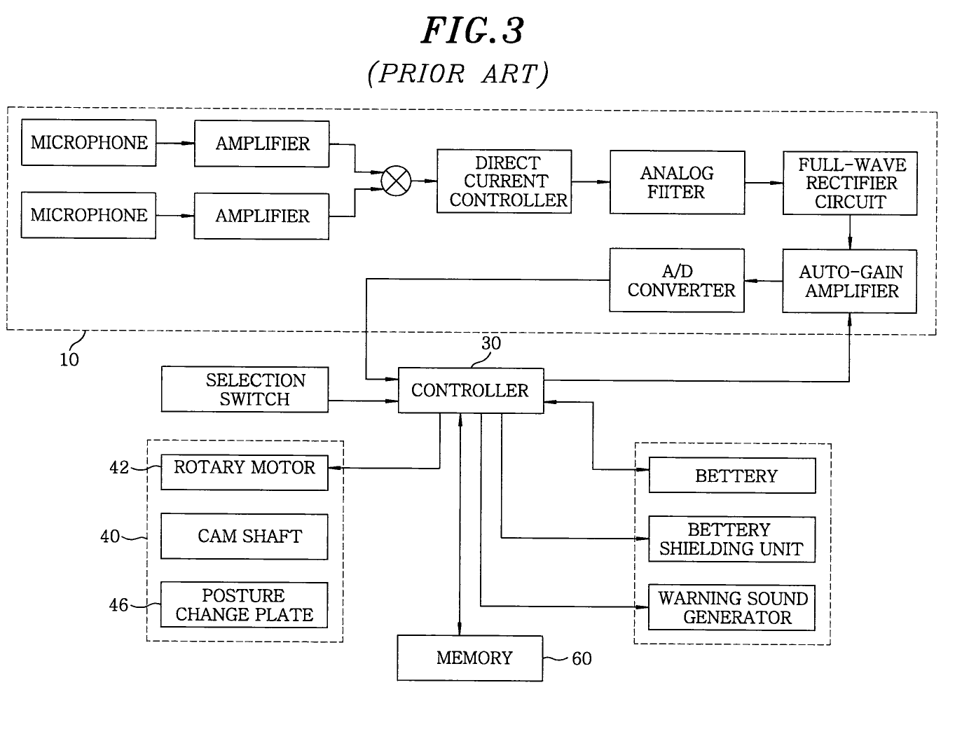

FIGS. 4 to 6 are diagrams illustrating another prior art snoring preventing apparatus;

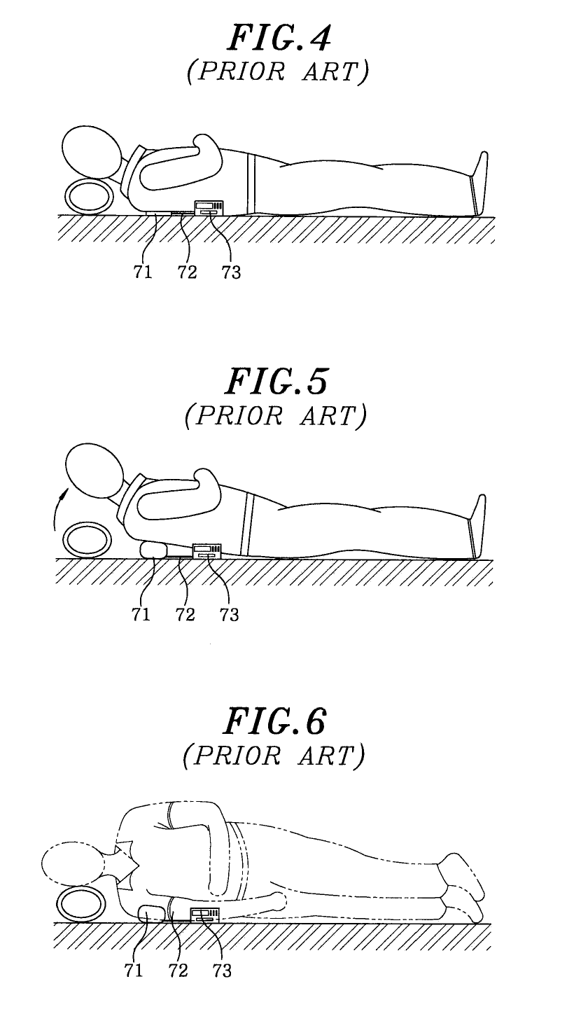

FIG. 7 is a diagram illustrating the configuration of clothes for preventing a sleeping respiratory obstruction and/or a decubitus according to an embodiment of the invention;

FIG. 8 is a front view illustrating the clothes for preventing a sleeping respiratory obstruction and/or a decubitus according to the embodiment of the invention;

FIG. 9 is a rear view illustrating the clothes for preventing a sleeping respiratory obstruction and/or a decubitus according to the embodiment of the invention;

FIG. 10 is a cross-sectional view taken along the line VII-VII of FIG. 9;

FIG. 11 is a flowchart illustrating a sleeping respiratory obstruction prevention method according to an embodiment of the invention;

FIG. 12 is a flowchart illustrating a decubitus prevention method according to another embodiment of the invention; and

FIGS. 13 to 15 are state diagrams explaining the operation of the clothes for preventing a sleeping respiratory obstruction and/or a decubitus according to the embodiments of the invention.

### BEST MODE FOR CARRYING OUT THE INVENTION

Hereinafter, embodiments of the present invention will be described in detail with reference to the accompanying drawings.

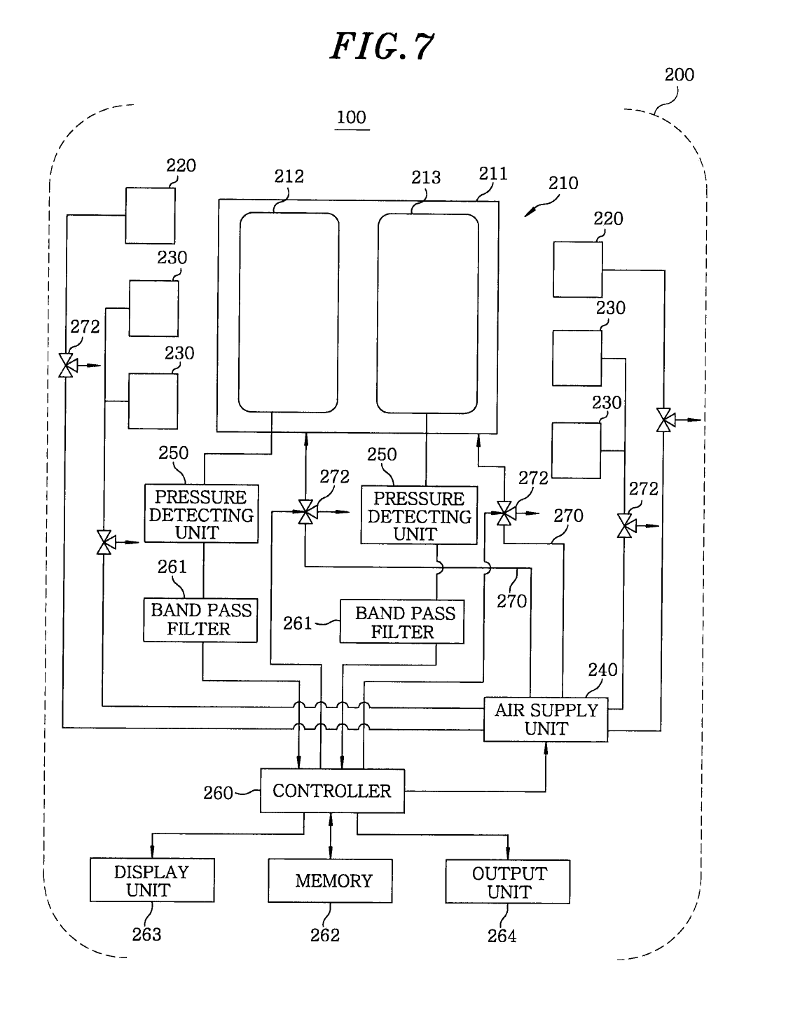

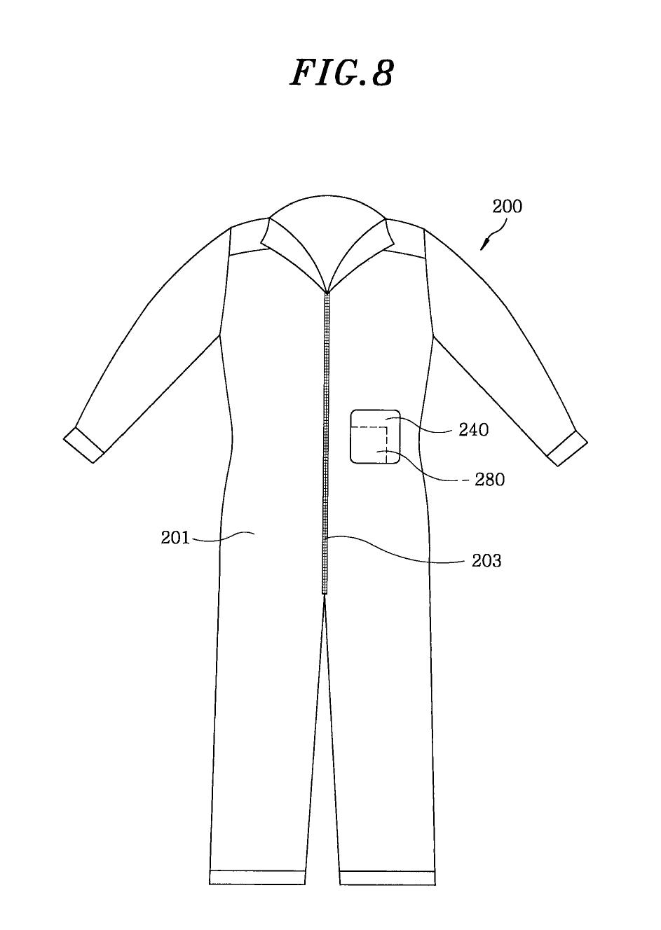

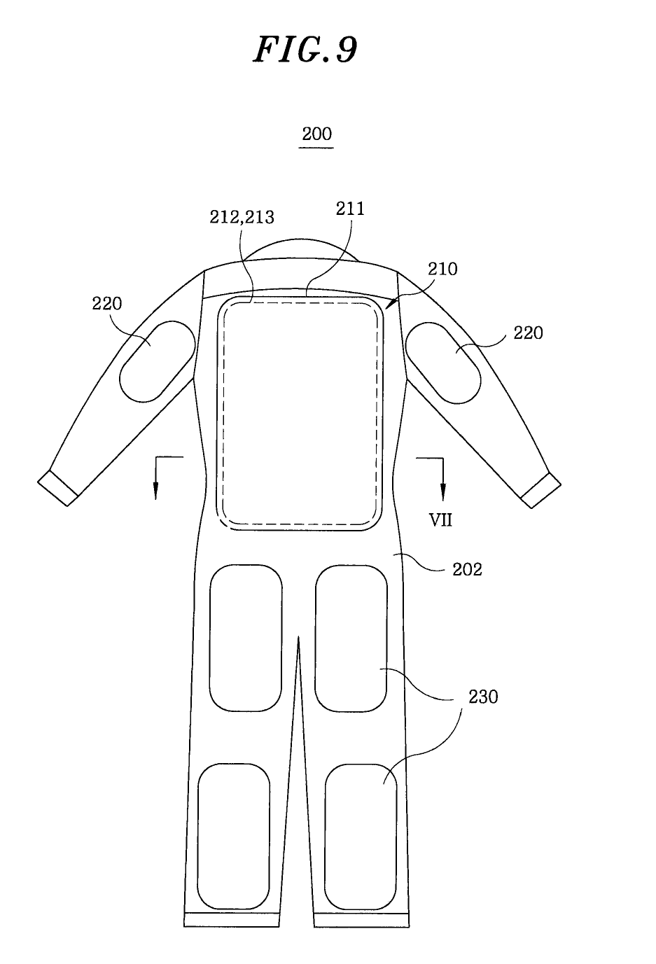

FIG. 7 is a diagram illustrating the configuration of clothes having a capability of preventing a sleeping respiratory obstruction and/or a decubitus according to an embodiment of the invention; and FIGS. 8 and 9 are a front and rear views illustrating the clothes shown in FIG. 7, respectively.

As shown in FIG. 7, clothes 200, which are worn by a sleeper or a patient, primarily include an apparatus 100 for preventing a sleeping respiratory obstruction and/or a decubitus. The apparatus 100 includes a plurality of air chambers 210, 220, and 230 provided at the rear of the clothes; an air supply unit 240 for supplying air to the air chambers 210, 220, and 230; a pressure detecting unit 250 for measuring the pressure of the air chamber 210; and a controller 260 for receiving the measured pressure from the pressure detecting unit 250 and controlling an air supply unit 240.

Referring to FIGS. 8 and 9, the clothes 200 adopt a design of a wearable unit while considering wearability and usability from a number of different design angles. Further, a functional structure and a suitable material selection are essential in such a wearable design. In particular, a material for regulating human body from harmful factors such as pleasantness, stability, electromagnetic wave shielding function, static electricity prevention function, insulation function and the like, are controlled can be selected.

In order to carry out a temperature control function, the clothes 200 not only discharges the sweat excreted during sleep through the material, but is also configured to have air holes in all directions of the body or in the armpit region where sweat can come out easily, so that the heat or humidity within the clothes can be discharged quickly, thereby improving the wear comfort. And, it is necessary to consider a sewing method of enabling for minimizing the friction between the material and the human body. In this regard, the clothes 200 for a user, e.g., a sleeper or a patient may be made of health oriented materials (e.g., chitosan fiber, silver fiber, bamboo fiber or the like, hi-tech materials such as aqua-trans, cool max mesh or the like), or environment friendly materials (e.g., organic cotton, tencel, natural mineral ion health fiber or the like).

The clothes 200 may be configured in a one-piece form, such as a flying suit, such that a sleeper or a patient can wear the clothes 200 all over. The clothes 200 include a front portion 201 and a rear portion 202 that cover the body, arms, and legs. The clothes 200 have a shape of having front open such that the user can easily wear the clothes 200. In the front open, a zipper 203 is provided to separate the clothes 200.

Alternatively, the clothes 200 may have a shape having side open. In addition, the zipper 203 is just an example, and various combining and disjoining units other than the zipper 203, such as gap adjustable fasteners, rubber bands, and snap buttons, may be used in conjunction with the same or a different kind.

The air chambers 210, 220, and 230 are provided at the rear of the clothes 200, that is, at the rear portion 202 to be shrunk and expanded. The air chambers 210, 220, and 230 are expanded by the air supplied from the air supply unit 240 and are made in a structure or/and of a material that can be shrunk and expanded. Various shapes including a circle and a polygon may be used for the air chambers, and more air chambers may be provided. In such a structure, the air chambers 210, 220, and 230 may include a portion that can be sheared or darted. Further, in view of a material, the air chambers 210, 220 and 230 may be made of, e.g., synthetic resin, natural rubber, artificial rubber, or fiber, which can be expanded or shrunk. The air chambers 210, 220, and 230 can have a sufficient volume to change the posture of the sleeper, e.g., from a supine position to a lateral position.

The air chambers 210, 220, and 230 are positioned to incline the user to one side at the rear of the clothes 200 so as to easily and naturally change the sleeper posture from the supine position to the lateral position when expanded. More specifically, a first air chamber 210 is provided at the rear portion 202 of the clothes 200 to correspond to the back of the user when he/she wears the clothes 200, a pair of second air chambers 220 is provided at positions corresponding to the arms of the user, and third air chambers 230 is provided at positions corresponding to the thighs and calves of the user in pairs. The first, second, and third air chambers 210, 220, and 230 change the user posture from the supine position to the lateral position of the left side or right side when expanded. The first, second, and third air chambers 210, 220, and 230 are connected to the air supply unit 240 via air supply lines 270.

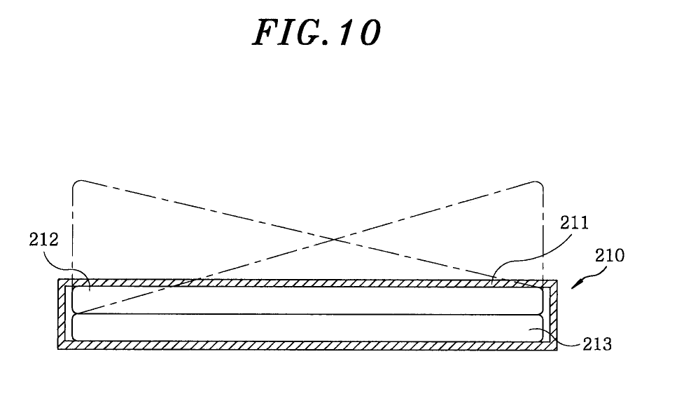

Referring to FIG. 10, there is shown a cross-sectional view taken along the line VII-VII of FIG. 9. As shown in FIG. 10, the first air chamber 210 includes a rectangular main body 211 having an area similar to the back of the user, and first and second air cells 212 and 213 vertically laminated in the main body 211. The first and second air cells 212 and 213 are alternately expanded in opposing right triangle shapes when the controller 260 receives the signal from the pressure detecting unit 250 and detects a sleeping respiratory obstruction or each time a predetermined time elapses, such that the user posture is changed to the lateral position facing a direction different from a previous position.

For example, the first air cell 212 is first expanded in a shape similar to a right triangle with a right-descending hypotenuse so as to change the user posture to the lateral position of the left side or right side, and the second cell 213 below the first air cell 212 is then expanded in a shape similar to a right triangle with a right-ascending hypotenuse.

Referring back to FIG. 7, the air supply unit 240 selectively supplies the air to the first, second, and third air chambers 210, 220, and 230 via the air supply lines 270 according to a control signal of the controller 260. Here, the air may be provided in a state of a compressed gas. In addition, an air pump may be used to supply the air through pumping.

The pressure detecting unit 250 may be implemented with a pressure measuring sensor. The pressure detecting unit 250 is preferably provided in the first air cells 212 and 213 of the first air chamber 210, which is most pressurized by the weight of the user, but it may be provided in the second and third air chambers 220 and 230. The pressure detecting unit 250 measures a pressure change in the first air chamber 210, the pressure change being caused by the heart beat of the user, e.g., a sleeper, the breathing, and the like of the user. It also measures the pressure of the first air chamber 210 pressurized by the weight of the user, and outputs the measured pressure to the controller 260 as an electrical signal. Meanwhile, to measure the pressure with the pressure detecting unit 250, the first and second air cells 212 and 213 in the first air chamber 210 are required to be filled with air, thereby exerting an enough volume required for pressure measurement. Here, the filled air may be an amount with which the user does not feel discomfort when lying down in the supine position.

The controller 260 receives the measured pressure signal from the pressure detecting unit 250. Further, upon sensing a snoring or a sleep apnea, the air supply unit 240 is configured to supply air to the first, second, and third air chambers 210, 220, and 230 in order to change the posture of the sleeper, e.g., from the supine position to the lateral position, thereby opening the upper airway of the sleeper, which in turn corrects the disordered breathing of the sleeper. As illustrated in the embodiment of the invention, the controller 260 receives the measured pressure signal from the pressure detecting unit 250 and alternately expands the first, second, and third air chambers 210, 220, and 230 in order to put the user in the lateral position facing a direction different from a previous position each time the snoring or the sleep apnea is detected.

Alternatively, when a user, e.g., a patient cannot make a posture change freely, if he/she is in the same posture for a predetermined time, the controller 260 receives a signal from the pressure detecting unit 250, which measures the pressure of the same part, and alternately expands the first, second, and third air chambers 210, 220, and 230 in order to put the sleeper in the lateral position facing a direction different from a previous position.

The controller 260 filters the signal transmitted from the pressure detecting unit 250 into a plurality of frequency bands by using a plurality of band pass filters 261, and calculates any one of the number of breathings, the number of heart beats, the number of snores, and the number of apneas of the sleeper, the time elapsed, and the like or the combination thereof from each of the frequency bands.

For example, the controller 260 receives the changed pressure of the first air chamber 210 from the pressure detecting unit 250 as an electrical signal. Further, the controller 260 calculates the number of breathings and the number of heart beats by analyzing waveform cycles thereof in the 0.1-0.5 Hz band and the 0.7-2.0 Hz band, which are obtained by the filtering of the band pass filter 261, and calculates the number of snores or sleep apneas based on the number of waveforms in a band over 50 Hz, all of which constitute a bio-signal of the user. Such a frequency band can vary in numerical value depending on a physical condition of the sleeper, a clinical trial method, and the like. Particularly, the presence or the absence of the snoring can be determined by observing a frequency characteristic, which is based on the result obtained in the clinical trial by use of an STFT (Short Time Fourier Transformation) method. In addition, the sleep apnea generally refers to a situation where a person does not breathe for longer than about 10 seconds or so, and such a situation occurs 5 or more times in an hour. The embodiment of the invention is to prevent the sleep apnea by encouraging a posture change when such a sleep apnea occurs. Therefore, the sleep apnea is determined to be present when no breathing is detected for 10 seconds.

Meanwhile, in order to store any one of the number of breathings, the number of heart beats, the number of snores, and the number of sleep apneas calculated by the controller 260 or the combination thereof as data, and to store the time elapsed according to a change in the measured pressure from the pressure detecting unit 250, the apparatus 100 further includes a memory 262. Further, the apparatus 100 additionally includes a display unit 263 which may be provided to externally display any one of the maximum number of breathings per minute, the minimum number of breathings per minute, the average number of breathings per minute, the cumulative number of snores, the combination thereof, or the time elapsed based on a selection signal from the data stored in the memory 262, for example, a manipulation signal given by the user through a control box 208 (see, FIG. 8). Furthermore, the apparatus further includes an output unit 264, such as a USB (Universal Serial Bus) port and the like, to transmit the data stored in the memory 262 to an external device, for example, a personal computer, a medical device, an analytical device, and the like.

The controller 260 can control the air supply unit 240 to directly shrink as well as expand the first, second, and third air chambers 210, 220, and 230. For example, if the air supply unit 240 is an air pump, the shrinkage and expansion are achieved through a forward rotation and a reverse rotation by the pump. In addition, although it is not limited thereto, as in the embodiment of the invention, three-way valves 272, which are controlled by the controller 260, are provided on routes, i.e., the air supply lines 270, respectively, through which the air is supplied from the air supply unit 240, such that the expanded first, second, and third air chambers 210, 220, and 230 can discharge the air for shrinkage.

The three-way valves 272 may be solenoid valves that can be operated by control signals from the controller 260, for example.

On the other hand, the air supply unit 240, the controller 260, the memory 262, the display unit 263, and the like may be provided to be separated from the clothes 200, but may also be separately attached thereto for the sake of a convenient use and in order to minimize the interference with the user. Here, the controller 260, the memory 262, the display unit 263, the air supply unit 240, and the like may be provided within the control box 280, of which the outer surface has a plurality of manipulation buttons (not shown) and attached to the clothes 200, and the air supply unit 240, the controller 260, and the like is provided by an easy-to-use rechargeable battery.

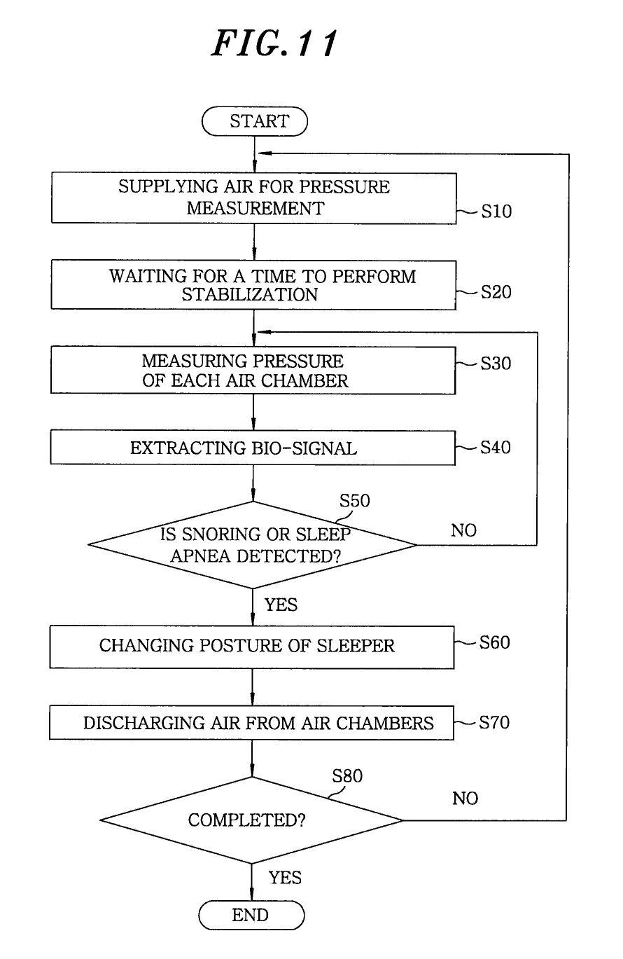

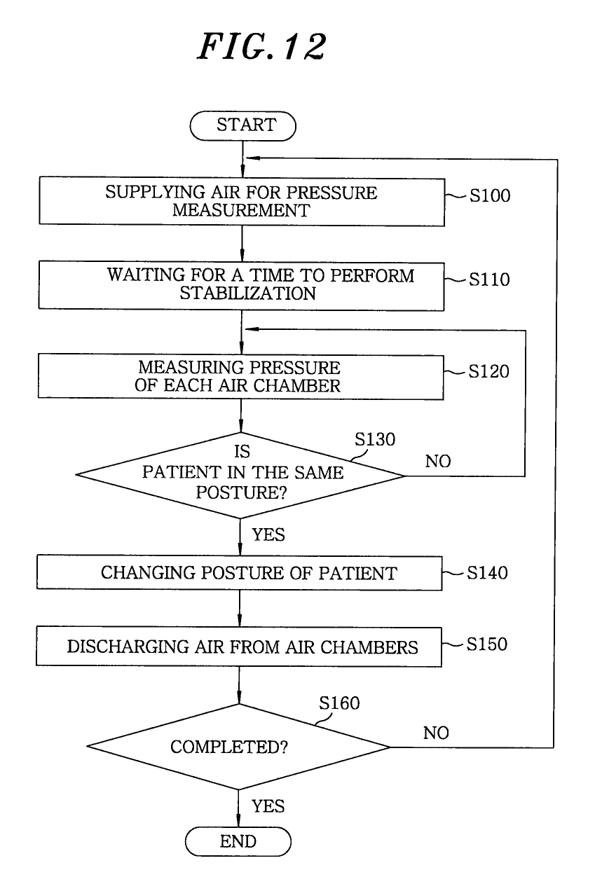

A sleeping respiratory obstruction and/or decubitus prevention method using the clothes 200 according to the embodiment of the invention will be described with reference to FIGS. 11 and 12.

First of all, FIG. 11 shows a flowchart illustrating a sleeping respiratory obstruction prevention method according to an embodiment of the invention.

The method, in which a user, i.e., a sleeper, is sleeping while wearing the clothes 200, includes the steps of: supplying air for pressure measurement to the air chambers (step S10); measuring the pressure of each of the air chambers (step S30); extracting a bio-signal from the measured pressure (step S40); determining whether or not the snoring or the sleep apnea is detected in the bio-signal (step S50); and changing the posture of the sleeper if the snoring or the sleep apnea is detected (Step S60).

In step S10, if the apparatus 100 is configured to be started when the sleeper lies down straight while wearing the clothes 200, under the control of the controller 260, the air with which the pressure of each of the air cells 212 and 213 of the first air chamber 210 can be measured is supplied to the extent that the sleeper does not feel discomfort while lying down in the supine position.

Meanwhile, in order to accurately measure the pressure in the first air chamber 210 after the step S10, a step S20 of waiting a time for stabilization of the first air chamber 210 is performed. That is, a time delay is added during which no pressure is measured for a predetermined time, e.g., 15-25 seconds or preferably about 20 seconds to stabilize the first air chamber 210.

In step S30, the pressure of each of the first and second air cells 212 and 213 pressurized by the weight of the sleeper is measured by the pressure detecting unit 250 after the step S20. Here, the duration of measurement is about 5 to 15 seconds, and preferably about 10 seconds. The pressure detecting unit 250 detects a pressure change within the first air chamber 210 based on the heart beat, the breathing, or the like of the sleeper and outputs the pressure change to the controller 260 as an electrical signal.

In the step S40, the electrical signal for the pressure value measured by the pressure detecting unit 250 is filtered by one or the band pass filters 261 for each frequency band. In addition, the bio-signal including the number of breathings, the number of heart beats, the number of snores, and the number of apneas is calculated or the combination thereof is calculated based on each frequency band, as described above. Here, the presence or the absence of the snoring can be determined by observing a frequency characteristic based on the result obtained in the clinical trial by use of an STFT (Short Time Fourier Transformation) method. Furthermore, the sleep apnea generally refers to a situation where a person does not breathe for longer than about 10 seconds or so and such a situation occurs 5 or more times in an hour. The embodiment of the invention is to prevent the sleep apnea by encouraging a posture change when such a sleep apnea occurs. Therefore, the sleep apnea is determined to be present when no breathing is detected for 10 seconds.

Meanwhile, the extracted bio-signal can be stored in the memory 263, and externally displayed as a maximum value, a minimum value, an average, and the like thereof via the display unit 263. In addition, the bio-signal may be transmitted to an external device, such as a personal computer, a diagnostic device, and the like through the output unit 264.

In the step S50, the presence of the snoring or the sleep apnea is determined based on predetermined data associated with one or a plurality of living bodies. If it is determined that there is not the snoring or the sleep apnea, a control process returns back to the step s30 to repeatedly perform the following steps. However, if it is determined that the snoring or the sleep apnea is detected, a control process goes to the step S60 to perform changing the posture of the sleeper.

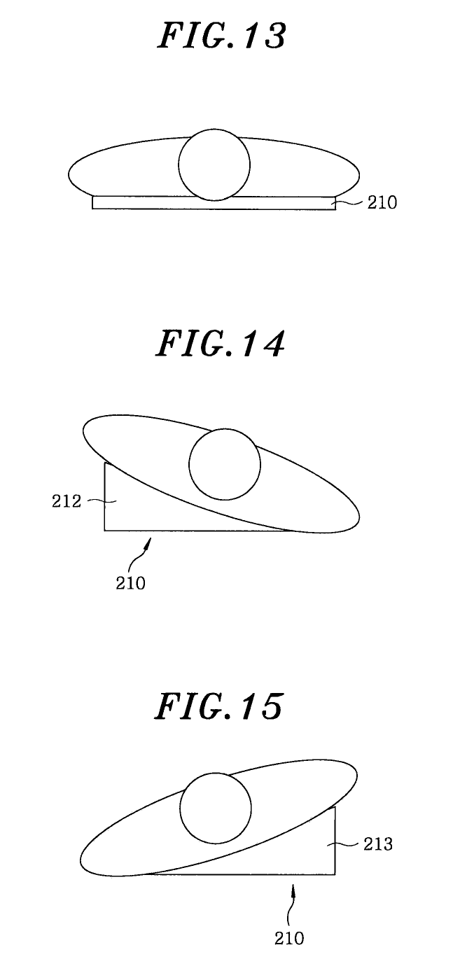

When the snoring or the sleep apnea is detected, the air supply unit 240 starts to supply the air to the first, second, and third air chambers 210, 220, and 230, thereby expanding the first, second, and third air chambers 210, 220, and 230. As in the embodiment of the invention, in case that the user lies down straight, as shown in FIG. 13, the air is supplied to the first air cell 212 of the first air chamber 210, and the second air chamber 220 and the third air chambers 230 provided at an arm and a leg corresponding to half of the body, thereby changing the posture of the sleeper from the supine position (shown in FIG. 13) to the lateral position (shown in FIG. 14), which in turn opens the upper airway correcting the disordered breathing of the sleeper.

In the step S60, if a predetermined time, e.g., 5 to 10 minutes, elapses after the posture of the sleeper is changed, a control process advances to the step S70 where the air of the first, second, and third air chambers 210, 220, and 230 is discharged so that the posture of the sleeper can return to the normal position. Here, the air discharge of the first, second, and third air chambers 210, 220, and 230 may be performed by the reverse operation of the air supply unit 240. However, in this embodiment, the air discharge may be made by the three-way valves 272, which operate based on the control signal from the controller 260, in order to naturally bring the sleeper to the normal position through a smooth air discharge.

Meanwhile, in step S70, if the sleeper returns to the supine position by the air discharge of the air chamber, as shown in FIG. 13, a control process goes to step S80 where if it is determined whether or not the usage is completed.

In step S80, if it is still in use, the steps following the step S10 are performed repeatedly.

Then, when the snoring or the sleep apnea is detected in step S50, the air is supplied to the second air cell 213 and the second and third air chambers 220 and 230 different from the previous one, thereby changing the posture to the lateral position of the opposite direction, as shown in FIG. 15.

That is, in the step S60 of changing the posture of the sleeper, the first, second, and third air chambers 210, 220, and 230 are alternately expanded to achieve a different lateral position.

According to the embodiment of the invention as above, the presence of the snoring or the sleep apnea by the sleeper can be accurately determined by the pressure change within the firs air chamber 210 pressurized by the weight, heart beat, breathing, and the like of the sleeper. In addition, when the snoring or the sleep apnea is detected, the sleep posture can be naturally changed from the supine position to the lateral position by selectively expanding the first, second, and third air chambers 210, 220, and 230 without awakening the sleeper so as to stop the snoring or the sleep apnea, thereby keeping the sleeper to have a sound sleep and preventing the disordered breathing, such as the snoring, the sleep apnea, and the like.

If the sleeper wears the clothes 200, the operation to change the posture of the sleeper for preventing the snoring or the sleep apnea is highly reliable even though there is a change in location and posture during sleep. Furthermore, the expanded first, second and third air chambers 210, 220, and 230 are shrunk to the original states, to thereby enable the sleeper to return to the comfortable posture. In addition, the first, second, and third air chambers 210, 220, and 230 are operated by the supply of the air, thereby restraining the noise generated upon the operation thereof to minimize the sleep disturbance.

Meanwhile, FIG. 12 shows a flowchart illustrating a decubitus prevention method according to another embodiment of the invention.

The method as shown in FIG. 8, in which a user (i.e., a patient) wears the clothes 200, includes: supplying the air for the pressure measurement to the air chambers (step S100); measuring the pressure of each of the air chambers (step S120); determining, based on the measured pressure, whether or not the patient is in the same posture for a predetermined time (step S130); and supplying the air to the air chambers to expand the air chambers if the patient is in the same posture for the predetermined time, to thereby change a posture of the patient (step S140).

In step S100, if the apparatus 100 is configured to be started when the patient lies down straight while wearing the clothes 200, the air with which the pressure in the first air chamber 210 can be measured is supplied to the extent that the patient does not feel discomfort while lying down in the supine position.

Meanwhile, in order to accurately measure the pressure of the first air chamber 210 after the step S100, a step S110 of waiting a time for stabilization of the first air chamber 210 is performed. That is, a time delay is added during which no pressure is measured for a predetermined time, e.g., 15-25 seconds or preferably about 20 seconds to stabilize the first air chamber 210.

In step S120, the pressure of the first air chamber 210 pressurized by the weight of the patient is measured by the pressure detecting unit 250. Here, the measured pressure value is transmitted to the controller 260.

In the step S130, if the measured pressure by the pressure detecting unit 250 is constant and unchanged for a predetermined time, e.g., 5 to 10 minutes, the controller 260 determines that the posture of the patient is required to be changed.

The time elapsed for the predetermined time may be stored in the memory 262 and externally displayed through the display unit 263, if necessary. In addition, the data therefor may be transmitted to the personal computer, the diagnostic device, and the like through the output unit 264.

According to the determination result in step S130, the step S140 of changing the posture of the patient is carried out.

The air supply unit 240 starts to supply the air to the first, second, and third air chambers 210, 220, and 230, thereby expanding the first, second, and third air chambers 210, 220, and 230. Here, as in the embodiment of the invention, when the patient lies down straight, as shown in FIG. 13, the air is supplied to the first air cell 212 of the first air chamber 210, and the second air chamber 220 and the third air chambers 230 corresponding to left half of the body, thereby changing the posture of the patient from the supine position (shown in FIG. 13) to the lateral position (shown in FIG. 14), which in turn allows oxygen and nutritive elements to be supplied to a blood vessel of a predetermined part pressurized by unrelieved pressured.

In the step S140, if the predetermined time elapses after the posture of the patient is changed, a control process goes to step S150 where the air of the first, second, and third air chambers 210, 220, and 230 is discharged so that the posture of the patient can return to the normal position. Here, the air discharge of the first, second, and third air chambers 210, 220, and 230 may be performed by the reverse operation of the air supply unit 240. However, in this embodiment, the air discharge may be made by the three-way valves 272, which operate based on the control signals from the controller 260, in order to naturally bring the patient to the normal position through a smooth air discharge.

Meanwhile, in step S150, if the patient returns to the supine position by the air discharge of the air chamber, as shown in FIG. 13, a control process proceeds to step S160 where if it is determined whether or not the usage is completed.

In step S160, if it is still in use, a control process returns back to the steps following the step S100 of supplying the air for the pressure measurement to the first air chamber 210 to repeatedly perform the above operations. Then, the air is supplied to the second air cell 213 of the first air chamber 210, and the second and third air chambers 220 and 230 corresponding to right half of the body different from the previous one, thereby changing the posture to the lateral position of the opposite direction, as shown in FIG. 15. That is, in the step S140 of changing the posture of the patient, the first, second, and third air chambers 210, 220, and 230 are alternately expanded to achieve a different lateral position.

As described above, according to the another embodiment of the invention, whether or not the posture of the patient is changed can be accurately determined based on the pressure change within the first air chamber 210 pressurized by the weight of the patient. In addition, since the first, second, and third air chambers 210, 220, and 230 are selectively expanded, the posture of the patient can be naturally changed from the supine position to the lateral position, thereby preventing the decubitus.

Although the clothes for preventing a sleeping respiratory obstruction and/or a decubitus and a sleeping respiratory obstruction and/or decubitus prevention method using the same have been described in connection with the embodiments of the invention, the embodiments are not limitative, but illustrative. It should be understood by those skilled in the art that various changes and modifications may be made without departing from the scope of the invention as defined in the following claims.

1. Clothes having a capability of preventing sleeping respiratory obstruction and/or decubitus, which is worn by a user, the clothes comprising:
a plurality of air chambers provided at the rear of the clothes, the air chambers being capable of being shrunk and expanded;
an air supply unit for supplying air to each of the air chambers;
a pressure detecting unit for measuring the pressure of each of the air chambers to produce a measured pressure as an electrical signal; and
a controller for receiving the measured pressure signal from the pressure detecting unit to supply the air to the air chambers by using the air supply unit when a snoring or a sleep apnea of the user is detected or a predetermined time elapses in the same posture in order to change a posture of the user.

2. The clothes of claim 1, wherein the plurality of air chambers include:
a first air chamber provided at a position corresponding to the back of the user when the user wears the clothes;
second air chambers provided at positions corresponding to the arms of the user; and
third air chambers provided at positions corresponding to the legs of the user, and
wherein the air chambers are positioned to incline the user to one side at the rear of the clothes so as to change the posture of the sleeper from a supine position and a lateral position of a left side or right side when expanded.

3. The clothes of claim 2, wherein the first air chamber includes a plurality of air cells laminated therein, and
the controller receives the measured pressure signal from the pressure detecting unit, and when a sleeping respiratory obstruction is detected or a predetermined time elapses in the same posture, alternately expands the plurality of air cells in opposing right triangle shapes to change the posture of the user from the supine position to the lateral position of the left side or right side.

4. The clothes of claim 1, further comprising:
three-way valves, which are controlled by the controller, provided on routes, respectively, through which the air is supplied, such that the air chambers discharges the air.

5. The clothes of claim 1, wherein the controller derives a bio signal of the user from the measured pressure signal by analyzing waveform cycles of the measured pressure signal in plurality of frequency bands, thereby controlling the air supply unit to shrink or expand the air chambers.

6. The clothes of claim 5, wherein the bio signal includes the number of breathings, the number of heart beats, and the number of snores or sleep apneas in each of frequency bands, respectively.

7. A sleeping respiratory obstruction prevention method for preventing snoring or sleep apneas, in which a user wears clothes having a plurality of shrinkable and expandable air chambers, the method comprising the steps of:
supplying air for pressure measurement to the air chambers;
measuring the pressure of one or more the air chambers pressurized by a weight of the user;
extracting a bio-signal from the measured pressure;
determining whether or not a snoring or a sleep apnea is detected in the bio-signal; and
supplying the air to the air chambers to expand the air chambers if the snoring or the sleep apnea is detected in the bio-signal, to thereby change a posture of the user.

8. The method of claim 7, wherein the step of changing a posture of the user changes the posture of the user from a supine position to a lateral position of a left side or right side by the plurality of air chambers provided at the rear of the clothes.

9. The method of claim 8, further comprising the step of:
waiting for a predetermined time to stabilize the air chambers after the step of supplying the air for the pressure measurement.

10. The method of claim 8, wherein the step of extracting a bio-signal converts the measured pressure into an electrical signal, filters the converted electrical signal into one or a plurality of frequency bands, and extracts any one of the number of breathings, the number of heart beats, the number of snores, and the number of sleep apneas, or a combination thereof from each of the frequency bands.

11. A decubitus prevention method, in which a user wears clothes having a plurality of shrinkable and expandable air chambers, the method comprising the steps of:
supplying air for pressure measurement to the air chambers;
measuring the pressure of one or more the air chambers pressurized by a weight of the user;
determining, based on the measured pressure, whether or not the user is in the same posture for a predetermined time; and
supplying the air to the air chambers to expand the air chambers if the user is in the same posture for the predetermined time, to thereby change a posture of the user.

12. The method of claim 11, wherein the step of changing a posture of the user changes the posture of the user from a supine position to a lateral position of a left side or right side by the plurality of air chambers provided at the rear of the clothes.

13. The method of claim 11, wherein the step of changing the posture of the user alternately expands the plurality of air chambers symmetrically provided on the left and right sides of the clothes to achieve a different lateral position when the posture of the user is required to be changed.

14. The method of claim 11, further comprising the step of:
discharging the air of the air chambers to return the posture of the user to a normal position if a predetermined time elapses after the posture of the user is changed in the step of changing the posture of the user.
